(19) United States
(12) Patent Application Publication
(10) Pub. No.: US 2016/0143586 A1
(43) Pub. Date: May 26, 2016

(54) A STRAP FOR A PORTABLE PULSE MEASURING DEVICE AND A PORTABLE PULSE MEASURING DEVICE

(71) Applicant: PULSEON OY, Espoo (FI)

(72) Inventors: Jari NOUSIAINEN, Espoo (FI); Ilkka KORHONEN, Lempaala (FI); Jarkko SAUNAMAKI, Vantaa (FI); Jaakko HATTULA, Espoo (FI)

(21) Appl. No.: 14/895,390

(22) PCT Filed: Jun. 11, 2014
(86) PCT No.: PCT/FI2014/050471

§ 371 Date: Dec. 2, 2015

(30) Foreign Application Priority Data
Jun. 11, 2013 (FI) 20135640

(51) Int. Cl. A61B 5/00; A61B 5/024

(52) U.S. Cl. CPC A61B 5/6843; A61B 5/02438; A61B 5/02416

(57) ABSTRACT

A strap for a pulse measuring device, wherein the strap includes an indicator configured to indicate tightness of the strap. A portable pulse measuring device having a strap configured to fasten the portable pulse measuring device on a human; and a mechanical indicator configured to indicate tightness of the strap are also disclosed.

### CROSS-REFERENCE TO RELATED APPLICATIONS

This application is the U.S. national phase of PCT Application No. PCT/FI2014/050471 filed on Jun. 11, 2014, which claims priority to FI Patent Application No. 20135640 filed on Jun. 11, 2013, the disclosures of which are incorporated in their entirety by reference herein.

### TECHNICAL FIELD

The invention relates to a strap for a portable pulse measuring device and to a portable pulse measuring device comprising a strap.

### BACKGROUND

Pulse can be measured using, for example, portable pulse measuring devices. A portable pulse measuring device may measure pulse optically. Another possibility is to use a separate heart rate belt around one's chest, and the belt then transmits measured pulse signals wirelessly to a monitoring device, for example, attached on a wrist or to an application running in a mobile phone.

The optical pulse measurement is based on the fact that light is emitted by a light source towards body tissue and at least one detector is configured to detect the intensity of reflected light after propagation through the human body tissue. There are several challenges when measuring pulse optically. The optical measurement is based on light absorption changes caused by blood flow in a lighted area. If the shape of the lighted area changes during the measurement, for example, due to movement of the pulse measuring device, the measurement is disturbed. Thus, for example, movements of a hand and of a human cause errors to the measurement in many ways. In order to avoid problems in the measurement, the portable pulse device needs to be as stable as possible in relation to skin and needs to minimize mechanical changes in tissue area during movement.

There are many ways to address the above problems. One solution is to tighten a strap of pulse a measuring device more. The problem, however, is that a user may tighten the strap too much, which in turn is uncomfortable and prevents blood flow in tissue. In turn, too loose tightening of the strap allows the portable pulse measuring device to move too much in relation, for example, to a wrist and body tissue.

It is possible to solve the above problems by design, for example, by making the device light, by optimizing the friction between skin and the device by material selection, or by making the device or strap of the device wider. Although these aspects may alleviate the problems in some cases, the basic problem still remains—how to provide an optimal strap tightening for a strap of a pulse measuring device.

### SUMMARY

According to one aspect, there is provided a strap for a portable pulse measuring device. The strap comprises an indicator configured to indicate tightness of the strap.

In one embodiment the strap is a stretchable strap comprising a non-stretchable sliding part built-in in the stretchable strap, wherein the stretchable strap comprises a section through which the indicator on the non-stretchable sliding part is visible.

In one embodiment the strap is a stretchable strap comprising a stretchable sliding part built-in in the stretchable strap, the stretchable sliding part being less stretchable than the stretchable strap, wherein the stretchable strap comprises a section through which the indicator on the stretchable sliding part is visible.

In one embodiment the indicator is provided by knitting the strap such that it provides a meter showing the level of tightness of the strap.

According to another aspect, there is provided a portable pulse measuring device comprising a strap configured to fasten the portable pulse measuring device on a human; and a mechanical indicator configured to indicate tightness of the strap.

In one embodiment the mechanical indicator is arranged into a link part connected to a main body of the portable pulse measuring device and to which the strap is attached.

In one embodiment the link part comprises a window in which the mechanical indicator is movable to indicate the tightness of the strap.

In one embodiment the link part comprises a rotating part to which the strap is attached. The indicator is attached to the rotating part such the when the rotating part rotates, the indicator moves in the window.

In one embodiment the link part comprises a sliding part to which the strap is attached. The indicator is attached to the sliding part such that when the sliding part moves, the indicator moves in the window.

According to another aspect, there is provided a strap for a portable pulse measuring device, wherein the strap comprises an indicator configured to indicate tightness of the strap, wherein the strap is a stretchable strap comprising a non-stretchable sliding part built-in in the stretchable strap and wherein the stretchable strap comprises a section through which the indicator on the non-stretchable sliding part is visible.

According to another aspect, there is provided a strap for a portable pulse measuring device, wherein the strap comprises: an indicator configured to indicate tightness of the strap, wherein the strap is a stretchable strap comprising a stretchable sliding part built-in in the stretchable strap, the stretchable sliding part being less stretchable than the stretchable strap and wherein the stretchable strap comprises a section through which the indicator on the stretchable sliding part is visible.

According to another aspect, there is provided a strap for a portable pulse measuring device, wherein the strap comprises an indicator configured to indicate tightness of the strap, wherein the indicator is provided by knitting the strap such that it provides a meter showing the level of tightness of the strap.

According to another aspect, there is provided a portable pulse measuring device comprising a strap configured to fasten the portable pulse measuring device on a human; and a link part, wherein the strap is connected to the link part and wherein the link part comprises a mechanical indicator configured to indicate tightness of the strap.

According to another aspect, there is provided a portable pulse measuring device comprising a strap according to any of the above aspects to fasten the portable pulse measuring device on a human.

### BRIEF DESCRIPTION OF THE DRAWINGS

The accompanying drawings, which are included to provide a further understanding of the invention and constitute a part of this specification, illustrate embodiments of the invention and together with the description help to explain the principles of the invention. In the drawings:

FIG. 1A discloses an arrangement comprising a mechanical indicator configured to indicate tightness of a strap of a portable pulse measuring device according to one embodiment of the invention.

FIG. 1B discloses a cross-section view of the arrangement of FIG. 1A.

FIG. 2 discloses an arrangement comprising a mechanical indicator configured to indicate tightness of a strap of a portable pulse measuring device according to one embodiment of the invention.

FIG. 3 discloses a strap for a portable pulse measuring device according to one embodiment of the invention.

FIG. 4 discloses a strap for a portable pulse measuring device according to one embodiment of the invention.

### DETAILED DESCRIPTION

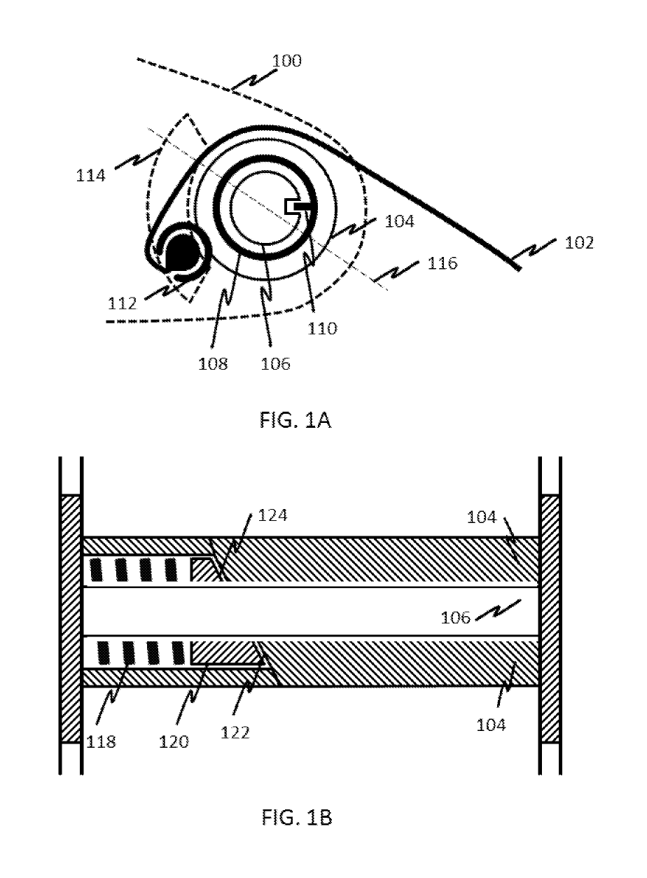

FIG. 1A discloses an arrangement comprising a mechanical indicator configured to indicate tightness of a strap 102 of a portable pulse measuring device according to one embodiment of the invention.

A link part 100 of the portable pulse measuring device provides an attachment point to a strap 102. The link part 100 may be removably attachable to a main body (not shown) of the portable pulse measuring device, or alternatively, the link part 100 may be an integral part of the main body. The link part 100 includes a fixed axle 106. A sliding part and a resilient member, for example a spring 108, are arranged around the fixed axle 104. Instead of the spring, any other resilient member may be used. A rotation blocker 110 is attached to the sliding part 108 in order to prevent the sliding part 108 to rotate around the fixed axle 106. A rotating non-sliding part 104 is arranged as an outmost element and the strap 102 is in contact with the rotating non-sliding part 104 in a section of its circumference, as illustrated in FIG. 1A. Reference number 112 indicates that the strap 102 is attached to the rotating non-sliding part 104. The link part 100 includes a window 114, for example a hole, in which an indicator is movable and the location of the indicator in the hole 114 depends on the tightness of the strap 102 when a user of the portable pulse measuring device fastens the device, for example, around his wrist.

In one embodiment, the indicator is attached to the rotating non-sliding part 104 and thus it moves when the rotating non-sliding part 104 rotates. The indicator may be a peg which moves in the window 114. In another embodiment, the indicator is a plate movable in the window 114 and it comprises a scale. A pointer has been arranged in the link part 100. When the plate moves as a result of pulling the strap, the pointer points to a certain point in the scale on the plate.

As illustrated in FIG. 1A, the hole 114 may take a form of a slot which is radially arranged in relation to the fixed axle 106. Reference number 116 refers to a cross-section which is illustrated in more detail in FIG. 1B.

In one embodiment, the window 114 is a hole in the link part. In another embodiment, the link part 100 comprises transparent section through which the indicator can be seen. For example, the link part 100 may be partly or wholly made of plastic and it may be partly or wholly transparent.

FIG. 1B discloses a cross-section view of the arrangement of FIG. 1A. As illustrated in FIG. 1B the rotating non-sliding part 104 partially directly surrounds the fixed axle 106 and is arranged to be rotatable about the fixed axle 106. The remaining part of the fixed axle 106 not directly surrounded by the rotating non-sliding part 104 is occupied by a spring 118 and a non-rotating sliding part 120.

When the strap 102 is pulled the rotating non-sliding part 104 rotates around the fixed axle 106. An inclined surface 122 of the rotating non-sliding part 104 faces towards an inclined surface 124 of the sliding non-rotating part 120. When the rotating non-sliding part 104 rotates it presses the sliding non-rotating part 120, and due to the inclined surfaces 122 and 124, the sliding non-rotating part 120 moves and compresses the spring 118. Although not illustrated in FIG. 1B, this action also moves the indicator in the hole 114. When the pulling stops and the tightness of the strap 102 is reduced, the spring 118 presses the sliding non-rotating part 120 and the sliding non-rotating part 120 generates a rotating force for the rotating non-sliding part 104 and thus the rotating non-sliding part 104 turns to its relaxed position.

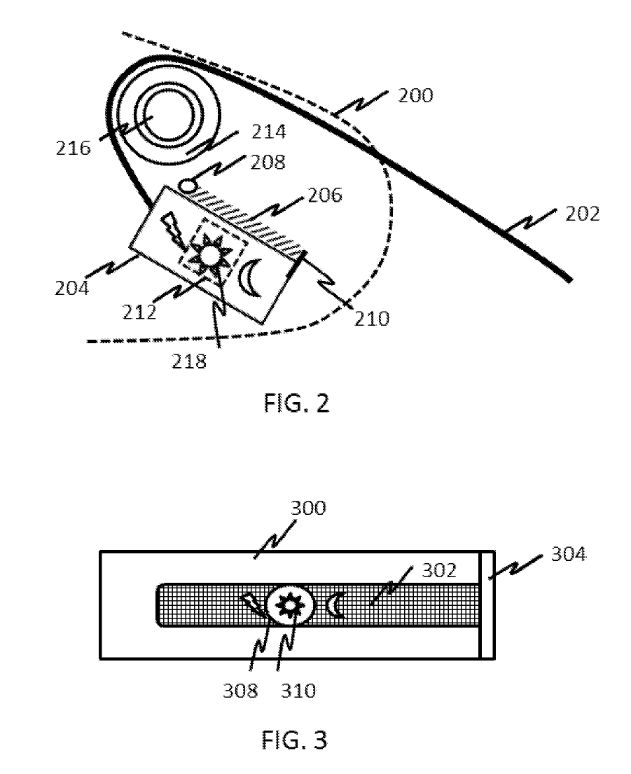

FIG. 2 discloses an arrangement comprising a mechanical indicator configured to indicate tightness of a strap 202 of a portable pulse measuring device according to one embodiment of the invention. Whereas FIG. 1A disclosed a turning force indicator FIG. 2 discloses a sliding force indicator.

A link part 200 of the portable pulse measuring device provides an attachment point to the strap 202. The link part 200 may be removably attachable to a main body (not shown) of the portable pulse measuring device or alternatively the link part 200 may be an integral part of the main body. The link part 200 includes a fixed axle 216. A rotating part 214 is arranged to be rotatably attached to the fixed axle 216. The strap 202 is arranged to be partially in contact with the rotating part 214 on the circumference of the rotating part 214, as disclosed in FIG. 2. A sliding part 204 is arranged in the link part 200 and the strap 202 is attached to the sliding part 204.

A first spring blocker 208 is arranged in the link part 200 and a second spring blocker is arranged in the sliding part 204. A spring 206 is arranged between the first spring blocker 208 and the second spring blocker 210. In one embodiment, the sliding part 204 comprises guiders which keep the sliding part 204 on its sliding track in the link part 200. When the strap 202 is pulled, the sliding part 204 moves and the spring 206 compresses. Instead of the spring 206, any other resilient member may be used.

The link part 200 also includes a window 212 through which an indicator 218 in the sliding part 204 or attached to the sliding part 204 can be seen. In one embodiment, the window 212 is a hole in the link part 200. In another embodiment, the link part 200 comprises a transparent section through which the indicator in the sliding part 204 or attached to the sliding part 204 can be seen. For example, the link part 200 may be partly or wholly made of plastic. Furthermore, it may be partly or wholly transparent.

As an example of the indicator, FIG. 2 discloses that the sliding part includes three patterns for indicating the tightness of the strap 202. Only one pattern can be seen in whole at a time through the window 212 in the link part 200. It is evident that FIG. 2 discloses only one example of a possible indicator. In another embodiment, an elongated slot may be arranged in the link part 200 and an indicator attached to the sliding part 204 moves in the elongated slot and indicates the current tightness of the strap 202.

FIG. 3 discloses a strap 300 for a portable pulse measuring device according to one embodiment of the invention. The strap 300 and a sliding part 302 are attached to a fixation part 304 via which they can be attached to a portable pulse measuring device body part. In the embodiment disclosed in FIG. 3, an indicator 310 indicating tightness of the strap 300 is included in the strap 300 itself. The strap 300 is stretchable. A sliding part 302 that is not stretchable, or has different stretching properties than the stretchable strap 300, is built-in in the stretchable strap 300. The sliding part 302 is configured in the stretchable strap 300 so that when the strap 300 stretches, the sliding part 302 remains unstretched. In other words, to achieve this functionality a cavity may be arranged in the strap 300 for the sliding part so that the sliding part 302 does not move when the strap 300 is stretched. The sliding part 302 has been equipped with one or more patterns, i.e. indicators 310, to indicate the tightness of the strap.

When the strap is pulled (i.e. when a user fastens the portable pulse measuring device comprising the strap, for example, onto his wrist and tightens the strap), the strap stretches and an indicator hole 308 moves in relation to the non-stretchable sliding part 302. A pattern indicating the tightness of the strap 300 is then visible via the hole 308.

In another embodiment of FIG. 3, the strap 300 is a stretchable strap comprising a sliding part 302 built-in in the stretchable strap 300. The sliding part 302, however, is stretchable but less stretchable than the stretchable strap 300. In other words, when the strap 300 is pulled (i.e. when a user fastens the portable pulse measuring device comprising the strap, for example, onto his wrist and tightens the strap), the strap 300 stretches, and at the same time, also the sliding part 302 stretches but less than the strap 300. An indicator hole 308 arranged in the strap 300 moves in relation to the sliding part 302, and an indicator 310 is visible through the indicator hole 308.

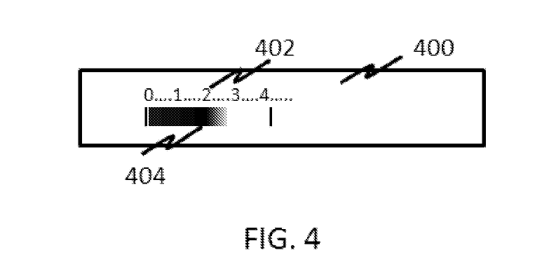

FIG. 4 discloses a strap 400 for a portable pulse measuring device according to one embodiment of the invention. As in the embodiment of FIG. 3, the strap 400 of the embodiment of FIG. 4 is stretchable. The strap 400 has been specially configured so that when the strap 400 is stretched, it shows a meter 404 showing the tension level of the strap 400. The strap 400 may include a numerical scale 402 or some other type of a scale or pattern to provide information about the tension/tightness of the strap 400. The meter 404 can be achieved, for example, by a special knitting of the strap 400 wholly or partially. When the strap 400 is stretched, the special knitting enables the meter 404 to be seen indicating the tension/tightness of the strap 400.

A benefit of the embodiments disclosed in FIGS. 1A, 1B and 2-4 is that the guided adjustment of tightness of the strap enables the use of the portable pulse measuring device in various operating situations. Moreover, the solution enables optimal strap tightness and avoids excessive loosening or tightening. Furthermore, the disclosed solution also takes into account physiological variations between individuals. Furthermore, the embodiments disclosed in FIGS. 1A, 1B and 2-4 are also advantageous when measuring pulse with pulse measuring devices that use optical pulse measurement techniques since undesired movements of the device may cause disturbances in the measurements. With the disclosed embodiments it is possible to ensure optimal strap tightness of the strap.

It is obvious to a person skilled in the art that with the advancement of technology, the basic idea of the invention may be implemented in various ways. The invention and its embodiments are thus not limited to the examples described above, instead they may vary within the scope of the claims.

1. A strap for a portable pulse measuring device, wherein the strap comprises:
an indicator configured to indicate tightness of the strap, wherein the strap is a stretchable strap comprising a non-stretchable sliding part built-in in the stretchable strap and wherein the stretchable strap comprises a section through which the indicator on the non-stretchable sliding part is visible.

2. The strap for a portable pulse measuring device, wherein the strap comprises:
an indicator configured to indicate tightness of the strap, wherein the strap is a stretchable strap comprising a stretchable sliding part built-in in the stretchable strap, the stretchable sliding part being less stretchable than the stretchable strap and wherein the stretchable strap comprises a section through which the indicator on the stretchable sliding part is visible.

3. The strap for a portable pulse measuring device, wherein the strap comprises:
an indicator configured to indicate tightness of the strap, wherein the indicator is provided by knitting the strap such that when the strap is stretched, the knitting enables a meter to be seen on the strap, wherein the meter alone indicates the tightness of the strap.

4. A portable pulse measuring device comprising a strap as claimed in claim 1, to fasten the portable pulse measuring device on a human.

5. A portable pulse measuring device comprising:
a main body comprising a link part;
a strap configured to fasten the portable pulse measuring device on a human; and
wherein the strap is connected to the link part and wherein the link part comprises a mechanical indicator configured to indicate tightness of the strap.

6. The portable pulse measuring device as claimed in claim 5, wherein the link part comprises a window in which the mechanical indicator is movable to indicate the tightness of the strap.

7. The portable pulse measuring device as claimed in claim 6, wherein the link part comprises a rotating part to which the strap is attached, and wherein the indicator is attached to the rotating part such that when the rotating part rotates, the indicator moves in the window.

8. The portable pulse measuring device as claimed in claim 6, wherein the link part comprises a sliding part to which the strap is attached, and wherein the indicator is attached to the sliding part such that when the sliding part moves, the indicator moves in the window.

9. The portable pulse measuring device comprising a strap as claimed in claim 2, to fasten the portable pulse measuring device on a human.

10. The portable pulse measuring device comprising a strap as claimed in claim 3, to fasten the portable pulse measuring device on a human.
